(19) United States
(12) Patent Application Publication
(10) Pub. No.: US 2010/0290989 A1
(43) Pub. Date: Nov. 18, 2010

(54) COMPOSITIONS AFFECTING HYALURONIC ACID MEDIATED ACTIVITY

(75) Inventors: Cornelia Tolg, London (CA); Eva Turley, London (CA); Jim McCarthy, St. Paul, MN (US); Leonard G. Luyt, London (CA)

Correspondence Address: KNOBBE MARTENS OLSON & BEAR LLP, 2040 MAIN STREET, FOURTEENTH FLOOR, IRVINE, CA, 92614, US

(73) Assignees: LONDON HEALTH SCIENCES CENTRE RESEARCH INC., London, ON (CA); REGENTS OF THE UNIVERSITY OF MINNESOTA, St. Paul, MN (US)

(21) Appl. No.: 12/682,774

(22) PCT Filed: Oct. 10, 2008
(86) PCT No.: PCT/CA08/01793

§ 371 Date: Aug. 5, 2010

Related U.S. Application Data
(60) Provisional application No. 60/960,756, filed on Oct. 12, 2007.

(51) Int. Cl. A61K 51/00; A61K 49/00; G01N 33/574; G01N 33/566; A61K 38/00; A61K 31/715; C07K 2/00; C07H 3/00; A61P 35/00

(52) U.S. Cl. 424/1.73; 424/9.1; 435/7.23; 436/501; 514/19.3; 514/54; 530/300; 536/123.1

(57) ABSTRACT

Compositions comprising hyaluronic acid (HA) or agents that interfere with HA binding of HA receptors and methods of using the same are provided. For example, there is provided a composition comprising HA oligomers ranging in size from 10-mer to 80-mer and use of the same for promoting migration, growth or survival of wild-type or transformed/cancerous cells. There is also provided a composition comprising an agent that interferes with binding between an HA oligomer of a size of about 10-mer to about 80-mer and an HA receptor. Such compositions may be useful for therapeutic, diagnostic or imaging applications. Examples of therapeutic use are wound repair or cancer treatment.

### FIELD OF THE INVENTION

The present invention relates to a composition comprising hyaluronic acid (HA) or agents that interfere with HA binding of HA receptors and methods of using the same.

### BACKGROUND OF THE INVENTION

Hyaluronic acid (HA) is a large anionic polymeric carbohydrate that influences tissue form and function on the basis of both mechanical and biological properties [1-4]. HA consists of repeating disaccharide units of N-acetyl-glucosamine and B-glucuronic acid. The molecular weight of HA is physiologically heterogeneous and ranges from small fragments <1000 daltons (e.g. 4-mer and 5-mer where 1-mer=1 saccharide “unit” of approximately 190 daltons) up to >1,000,000 daltons (e.g. a 5208-mer). HA is important for maintaining tissue hydration, cushioning joints, preserving cell free space within specific tissues or regulation of cell behavior such as migration or proliferation via activation of cell signalling pathways. During development, HA involvement is known in many morphogenetic events such as neural crest cell migration, cardiac development and ductal branching of the prostate gland and mammary gland. In the adult, HA is also an important adhesion/migration substrate that regulates bone marrow stem cell trafficking and tissue response-to-repair.

HA is synthesized in mammals by one or more members of a family of three HA synthases (HAS1, 2, or 3) [5-7]. Degradation of HA occurs by the concerted action of both exoglycosidases that sequentially remove carbohydrates from the reducing end of the polymers and endoglycosidases (known as hyaluronidases or HYaI) that cleave HA polymers into relatively large oligosaccharides [3]. These fragments may be internalized and degraded further, or they can stimulate angiogenesis, inflammation or tumor progression if released to the extracellular environs.

Cellular receptors for HA, along with specific extracellular HA-binding proteins and proteoglycans, can bind HA to retain and organize it to the cell surface or within the extracellular milieu [8,9].

HA-rich matrices can alter the growth, survival or migration of both normal and transformed cells. HA interactions with specific cell-associated hyaladherins such as CD44 and Rhamm, activate cell signalling pathways and are associated with enhanced responsiveness to growth and survival factors [4,10].

The first HA biomedical product, Healon, was developed in the 1970s and 1980s and is approved for use in eye surgery (i.e., corneal transplantation, cataract surgery, glaucoma surgery and surgery to repair retinal detachment). HA may also be used postoperatively to induce tissue healing.

HA is also injected into the knee joint to treat osteoarthritis of the knee. However, some placebo controlled studies have cast doubt on the efficacy of HA injections, and HA is recommended primarily as a last alternative to surgery.

HA is available as an oral supplement, although effectiveness is yet to be conclusively demonstrated. Some preliminary clinical studies exist that suggest that oral administration of HA has a positive effect on osteoarthritis.

Due to its high biocompatibility and its common presence in the extracellular matrix of tissues, HA is gaining popularity as a biomaterial scaffold in tissue engineering research and in other biomedical applications targeting these tissues.

In contrast to the proposed benefits of HA during wound repair and inflammatory processes, in some cancers, HA levels correlate well with malignancy and poor prognosis. HA is thus a possible tumor marker for prostate or breast cancer and may also be useful to monitor the progression of the disease.

Gene array analysis of synchronized cells indicates that transcripts for both HAS2 and the HA receptor Rhamm are increased at G2/M [1,1] and HA synthesis increases at this stage of the cell cycle [3, 12, 13]. Both HA retention to the cell surface and Rhamm function are required for progression through the G2M boundary of the cell cycle [12,14]. Blocking of these HA functions with very small (2500 dalton) HA oligosaccharides, or recombinant protein fragments of either CD44 or Rhamm inhibit the anchorage-independent growth and/or invasion of tumor cells in vitro and tumor formation in vivo [14-16]. Clinical relevance of these results from experimental models is suggested by reports that elevated levels of HA within the primary tumor are an independent negative prognostic indicator in prostate and breast cancer.

Primary prostate tumor progression is accompanied by significant increases in both HA deposition and hyaluronidase levels in the tumor-associated stroma and in the carcinoma, respectively [17-19]. Perineural infiltration, seminal vesicle invasion by tumors and PSA recurrence are all associated with a high intensity of stromal HA staining in prostate cancer patients undergoing radical prostatectomy [20]. The interplay of HA synthases and hyaluronidases results in the formation of HA-rich matrices with heterogeneous-sized polymers and fragments of HA facilitate tumor growth and angiogenesis [18, 19, 21, 22]. Retrospective analysis of human prostate (and other) tumor specimens has shown that an increased ratio of hyaluronidase:HA expression is an independent indicator of poor prognosis, consistent with the hypothesis that partially fragmented HA in the tumor microenvironment is associated with enhanced malignant potential [23, 24].

As prostate tumors progress to become metastatic, and/or acquire androgen independence following therapy, carcinomas may develop the ability to synthesize their own HA by multiple mechanisms [25].

Given, the correlation of HA with cancer, current HA containing formulations may contain components that enhance or promote certain cancers. Therefore, there is a need to develop a composition taking into consideration the varied biological effects of HA.

### SUMMARY OF THE INVENTION

An object of an aspect of the present invention is to provide an improved composition comprising an HA.

In accordance with an aspect there is provided a composition comprising a first HA oligomer of a size of about 10-mer to about 40-mer in higher concentration than any other size of HA oligomer.

In accordance with another aspect there is provided a method of promoting migration of a wild-type cell in a subject comprising administering an effective amount of a composition comprising a first HA oligomer of a size of about 10-mer to about 40-mer in higher concentration than any other size of HA oligomer to a subject in need of such treatment.

In accordance with yet another aspect there is provided a composition comprising a first HA oligomer of a size of about 40-mer to about 80-mer in higher concentration than any other size of HA oligomer.

In accordance with still another aspect there is provided a method of promoting migration of a cancerous cell in a subject comprising administering an effective amount of a composition comprising a first HA oligomer of a size of about 40-mer to about 80-mer in higher concentration than any other size of HA oligomer to a subject in need of such treatment.

In accordance with a further aspect there is provided a composition comprising a peptide that binds an HA oligomer of a size of about 10-mer to about 80-mer.

In accordance with an even further aspect there is provided a composition comprising a peptide that binds to an HA binding domain of an HA receptor.

In accordance with still a further aspect there is provided an HA oligomer having a size from 10 to about 40 saccharides.

In accordance with yet a further aspect there is provided an HA oligomer having a size from about 40 to about 80 saccharides.

Novel features of these and other aspects will become apparent to those of skill in the art upon examination of the following detailed description of the invention. It should be understood, however, that the detailed description of the invention and the specific examples presented therein, are provided for illustration purposes only and are not meant to be limiting.

### BRIEF DESCRIPTION OF THE DRAWINGS

The following drawings are illustrative only and are not meant to limit the scope of the claims:

FIG. 1 shows a scheme for isolating HA binding peptides using a phage display library and affinity chromatography;

FIG. 2 shows two independent determinations of peptide 15-1/HA binding constant by isothermal calorimetry;

FIG. 3a shows a comparison of the ability of a purified 30-mer HA oligomer to increase migration in Rhamm null (Rh−/−) fibroblasts and Rh−/− fibroblasts transfected with full length Rhamm cDNA (Rh-rescued) in a scratch wound assay;

FIG. 3b shows a comparison of the ability of a purified 30-mer HA oligomer to promote migration of wild-type (Wt) fibroblast cells and Rh−/− cells in a scratch wound assay in the presence of a peptide that specifically binds low molecular weight HA (avg. 10 000 Da) or a control scrambled peptide;

FIG. 4 shows a comparison of inhibition of anchorage-independent growth of prostate tumor cells in the presence of a peptide that specifically binds low molecular weight HA (avg. 10 000 Da) or a control scrambled peptide in a methylcellulose assay;

FIG. 5 shows a comparison of inhibition of in vivo tumor growth of prostate tumor cells injected subcutaneously into immuno-compromised mice, in the presence of a peptide that specifically binds low molecular weight HA (avg. 10 000 Da) or a control scrambled peptide; in a methylcellulose assay;

FIG. 6 shows a comparison of restoration of anchorage-independent growth of prostate tumor cells by addition of discrete sizes of exogenous HA oligomers in a methylcellulose assay;

FIG. 7 shows a comparison of the ability of various discrete sizes of exogenous HA oligomers to promote migration of prostate tumor cells in a scratch wound assay;

FIGS. 8a and 8b show a comparison of the ability of various discrete sizes of exogenous HA oligomers to promote migration of wild-type fibroblast cells in a scratch wound assay; and

FIG. 9 shows a comparison of the ability of a mixture of HA oligomers and a purified 30-mer oligomer to promote migration of wild-type fibroblast cells in a scratch wound assay in the presence of a peptide that specifically binds low molecular weight HA (avg. 10 000 Da) or a control scrambled peptide.

FIG. 10a shows that an HA binding peptide reduces scratch wound induced migration of human breast cancer cells.

FIG. 10b shows that an HA binding peptide reduces anchorage independent growth of human breast cancer cells.

### DETAILED DESCRIPTION

HA has been shown to have beneficial properties such as promotion of wound healing, but has also been correlated with cancer. HA appears to be able to alter the growth or survival of both wild-type and transformed/cancerous cells.

Provided herein, are findings of a relation between sizes of HA fragments and different biological processes. On the basis of these findings, compositions may be developed that distinguish between wild-type cells and transformed/cancerous cells with respect to promotion of migration, growth or survival of cells. Certain exemplary compositions, that are formulated based on these findings, comprise sizes of HA that have beneficial effects involving wild-type cells, for example promoting wound healing. Other exemplary compositions, formulated based on these findings, do not include sizes of HA that may be associated with promotion of migration, growth or survival of cancerous cells. Still other exemplary compositions, formulated based on these findings, comprise an agent that binds to sizes of HA that may be associated with promotion of migration, growth or survival of cancerous cells. Yet other exemplary compositions comprise an agent that binds to an HA binding domain of an HA receptor. Still further exemplary compositions, formulated based on these findings, comprise sizes of HA that may be associated with promotion of migration, growth or survival of cancerous cells, and may be used for diagnostic purposes.

HA sizes associated with promotion of migration, growth or survival of wild-type cells, will typically include HA sizes of 10-mer to 50-mer (ie., 10 to 50 saccharides). It will be understood that for certain therapeutic or diagnostic purposes described herein the upper and lower limit of the size range may be in between 10-mer to 50-mer. For example, HA having 10 to 40 saccharides, 10 to about 30 saccharides or 10 to 30 saccharides are contemplated. HA fragments of these sizes will typically preferentially promote migration, growth or survival of a targeted wild-type cell over a cancerous cell. A composition comprising HA sizes associated with migration, growth or survival of wild-type cells will typically comprise a size fraction of HA having a higher concentration of 10-mer to about 40-mer HA than other HA sizes or for example a higher concentration of 10-mer to about 35-mer than other HA sizes. In certain examples, a composition comprising HA sizes associated with migration, growth or survival of wild-type cells may comprise a size fraction of HA having a higher concentration of about 20-mer to about 35-mer HA than other HA sizes. In certain further examples, a composition comprising HA sizes associated with migration, growth or survival of wild-type cells may comprise a size fraction of HA having a higher concentration of about 20-mer to about 30-mer HA than other HA sizes. In still other examples, a composition comprising HA sizes associated with migration, growth or survival of wild-type cells may comprise a size fraction of HA having a higher concentration of about 10-mer to about 30-mer HA than other HA sizes. In yet other examples, a composition comprising HA sizes associated with migration, growth or survival of wild-type cells may comprise a size fraction of HA that preferentially promotes migration, growth or survival of a wild-type cell over a cancerous cell. In each of the examples in this paragraph, can be modified so that the largest concentration of HA is an HA having about 30 saccharides.

Compositions comprising HA sizes associated with migration, growth or survival of wild-type cells or HA sizes associated with preferential migration, growth or survival of wild-type cells over cancerous cells may be used in accordance with any convenient HA therapeutic treatment that involves migration of wild-type cells. Macromolecular solutions containing HA, for example, have been popular substances in the effort to prevent tissue adhesion and aid in wound healing. It is well known that undesired tissue damage results from most surgical procedures, where cutting, desiccation, ischemic, and manipulative abrasions occur. U.S. Pat. No. 4,141,973 discloses an ultra-pure HA fraction having an average molecular weight of at least about 750,000 that has, among its many uses, the prevention of scar tissue formation and adhesion following surgery by introducing an HA solution into the surgical site, either during surgery or postoperatively. U.S. Pat. No. 5,234,914 describes the use of an HA solution for the treatment of hemorrhoids and anorectal diseases which are accompanied by traumatized tissue. By adhering to the anorectal epithelium and rectal mucosa, the HA solution provides a reduction in the pain, burning, inflammation, itching, and swelling associated with the above causes. U.S. Pat. No. 5,190,759 describes the use of an HA solution, alone or in combination with dextran, for preventing adhesions between body tissues following surgical procedures. U.S. Pat. No. 5,409,904 describes the use of an HA solution, either alone or in combination with other viscoelastic substances, for preventing post-operative adhesions between healing tissues by introducing the solution into a surgical site during surgery or postoperatively. U.S. Pat. No. 5,681,825 describes the use of an HA solution into the site of the surgical operation, for facilitating surgical operations that involve the eye or eye area. U.S. Pat. No. 5,140,016 describes the use of HA based compositions and improved methods for preventing adhesions during surgery. Still other examples of therapeutic treatment will be recognized by the skilled person, where the cells targeted for therapy are wild-type cells or tissue, for example treatment of burns with HA, embedding sutures with HA, and the like.

HA sizes that may be associated with promotion of migration, growth or survival of cancerous cells, will typically include sizes of about 40-mer to about 100-mer as well as certain sizes that are greater than about 100000 daltons, for example an HA of 1145-mer (approximately 220000 daltons) or 4167-mer (approximately 790000 daltons). It will be understood that for certain therapeutic or diagnostic purposes described herein the upper and lower limit of the size range may be in between 40-mer to 100-mer. In certain examples, a composition may be formulated without HA sizes that promote migration, growth or survival of cancerous cells. For example, HA sizes of about 47-mer, about 1145-mer or about 4167-mer may be absent from such compositions. In certain other examples, a composition may comprise an agent that binds to HA sizes of about 47-mer, about 1145-mer or about 4167-mer. In still other examples, a composition may comprise HA sizes that promote migration, growth or survival of cancer cells, for example sizes of about 47-mer, about 1145-mer or about 4167-mer, for promoting mobilization of cells to a location where the cells would be more susceptible to therapeutic intervention. In yet further examples, a composition may comprise HA sizes that promote migration, growth or survival of cancer cells, for example sizes of about 47-mer, about 1145-mer or about 4167-mer, for promoting mobilization of cells in assays for cancer research or investigative purposes, for example drug screening assays. A composition comprising HA sizes associated with migration, growth or survival of cancerous cells will typically comprise a size fraction of HA having a higher concentration of about 40-mer to about 80-mer HA than other HA sizes or for example 40-mer to about 60-mer than other HA sizes.

Without wishing to be limited by theory, findings provided herein suggest that HA sizes of about 47-mer (approximately 9800 daltons) as well as certain sizes greater than about 100000 daltons, for example an HA of 1145-mer or 4167-mer, can promote migration, growth or survival of cancerous cells. Accordingly, diagnostic, imaging and therapeutic techniques are provided comprising use of an HA oligomer having a size associated with promotion or survival of cancerous cells. For example, a method for imaging cancer cells is provided comprising use of labeled HA oligomers. The HA oligomers may be metal-labelled or fluorophore-labelled, for example. Labelled HA oligomers may also be used in a method for diagnosing cancer. As another example, a method for treating cancer is provided comprising use of HA oligomers linked to a therapeutic anti-cancer compound, either directly or via a linker or carrier molecule.

The use of HA to image, diagnose or treat cancer has been described previously and the specific size ranges described herein may be used accordingly. For example, use of HA in combination with surgical or chemotherapeutic treatment of cancerous masses is described in US Patent Publication 20050113335 published May 26, 2005. One such possible treatment is the use of combinations of HA and liposomes and/or any suitable therapeutic agent which, for example, may be bound to HA. HA may be equally used as a targeting and delivery moiety for any suitable agent. See PCT. Application WO 91/04058. HA may also be infused to a patient to mobilize cancer cells from solid tissues into the blood where the cancer cells exist as single cell suspensions and are rendered more drug sensitive and/or are more effectively attacked by a therapeutic agent and/or removed by some physical procedure such as leukapheresis.

Any cancer type that increases migration or proliferation in response to HA may be treated using HA, HA binding peptides, or mimicking compounds thereof. Cancers include, but are not limited to, squamous cell carcinoma, fibrosarcoma, sarcoid carcinoma, melanoma, mammary cancer, lung cancer, colorectal cancer, renal cancer, osteosarcoma, cutaneous melanoma, basal cell carcinoma, pancreatic cancer, bladder cancer, brain cancer, ovarian cancer, breast cancer, prostate cancer, leukemia, lymphoma and metastases derived therefrom.

The source of HA is not critical. HA may be used in its naturally-occurring form, or may be chemically synthesized. Naturally-occurring HA may be obtained from any biological source, including extraction from tissue and biosynthesis. Extraction from tissue typically uses fresh or frozen cocks' combs (U.S. Pat. No. 5,336,767), although other tissues including the synovial fluid of joints (Kvam et al., Anal. Biochem. 211, 44-49 (1993)), human umbilical cord tissue, bovine vitreous humor, and bovine tracheae, have been used. It is also possible to prepare HA by microbiological methods, such as by cultivating a microorganism belonging to the genus Streptococcus which is capable of producing HA in a culture medium (U.S. Pat. Nos. 4,897,349; 4,801,539; 4,780,414; 4,517,295; 5,316,926). As a further example of an HA biological source, isolation of HA from eggshell membranes has been described in US Patent Publication 20040180851 published Sep. 16, 2004. The total HA content of eggshell membrane was estimated between 0.5%-10% using a uronic acid assay, while molecular weight range was estimated to be approximately 50,000-100,000 daltons using size exclusion chromatography and a refractive index detector. There are numerous examples of methods for isolating and/or purifying HA from biological sources, e.g., animal sources. Selected patent references describing methods to isolate and purify HA include U.S. Pat. Nos. 4,141,973; 4,784,990; 5,099,013; 5,166,331; 5,316,926; 5,411,874; 5,559,104 and 5,925,626.

Any suitable form or derivative of HA may be used. HA may be derivatized by any convenient procedure. For example, strategies have included esterification of HA (U.S. Pat. Nos. 4,957,744 and 5,336,767), acrylation of HA (U.S. Pat. No. 5,410,016), and cross-linking of HA using divinyl sulfone (U.S. Pat. No. 4,582,865) or glycidyl ether (U.S. Pat. No. 4,713,448). Other examples of HA derivatives are described in US Patent Publication 20020132790 published Sep. 19, 2002. The production and properties of representative HA fractions HYALECTIN and HYALASTINE are described in European Patent No. 0 138 572. Also useful may be partial and total esters of HA with various alcohols, including alkyl and arylalkylic alcohols, as described in U.S. Pat. Nos. 4,851,521 and 4,965,353, and the inter- and intramolecular esters of HA described in EP 0 341 745, wherein part or all of the carboxyl groups are esterified with hydroxyl groups of the same and/or different molecules of HA. Crosslinked esters of HA, as described in EP 0 265 116 may also be used. Methods of derivatizing HA or introducing side chains in HA are also described in US Patent Publication 20070149441 published Jun. 28, 2007. For example, a methodology for introducing side chains into HA by carbodimide-mediated coupling of primary or secondary amines to the carboxyl group of the glucuronic acid moiety using an active ester intermediate is described. This methodology may be used to generate HA with different terminal functional groups for crosslinking including acetals, aldehydes, amines, and hydrazides. A wide range of functionalized amines are commercially available allowing for a wide variety of different functional groups useful for crosslinking under physiological conditions using this methodology. Side chains need not be restricted to functional cross-linking groups; for example, side chains may be branched or unbranched, and be saturated or may contain one or more multiple bonds. The carbon atoms of the side chain may be continuous or may be separated by one or more functional groups such as an oxygen atom, a keto group, an amino group, an oxycarbonyl group, and the like. The side chain may be substituted with aryl moieties or halogen atoms, or may in whole or in part be formed by ring structures such as cyclopentyl, cyclohexyl, cycloheptyl, and the like. The side chain may be a bioactive peptide, e.g., containing receptor binding sites, crosslinking sites for transglutaminases, or proteolytic cleavage sites. Thus, it will be understood that any pharmaceutically acceptable form of HA may be used, including any pharmaceutically acceptable salt or derivative. Pharmaceutically acceptable forms of HA including HA per se, potassium HA, or sodium HA are merely popular examples of HA used in the art, and are not meant to be limiting for the purposes described herein. It will be understood that reference to HA includes HA as well as any pharmaceutically acceptable form thereof.

An agent that binds HA may be any type of molecule, naturally-occurring or synthetic, that is capable of binding HA and interfering with binding of HA to its HA receptors. For example, an agent that binds HA may be a peptide, small chemical, or polynucleotide that mimics an HA binding domain of an HA receptor. An example of a peptide that binds HA is a peptide comprising the sequence STMMSRSHKTRSHHV. Another example of a peptide that binds HA is a peptide comprising a structural motif of F/WHKP/A. An agent that binds HA may be used in the treatment or prophylaxis of cancer, for example by binding to HA fragment sizes that may be associated with promotion of migration, growth or survival of cancerous cells.

HA binding peptides may be prepared by conventional techniques such as chemical synthesis using techniques well known in the chemistry of proteins such as solid phase synthesis (Merrifield, J. Am. Chem. Assoc. 85:2149-2154 (1964)) or synthesis in homogenous solution (Houbenweyl, Methods of Organic Chemistry, ed. E. Wansch, Vol. 15 I and II, Thieme, Stuttgart (1987)).

As another example of peptide preparation, HA binding peptides may be produced by recombinant DNA technology. To prepare the peptides of the invention by recombinant DNA techniques, a DNA sequence encoding the HA-binding peptide must be prepared. An expression vector comprising a DNA molecule encoding a HA-binding peptide may be adapted for transfection or transformation of a host cell. The nucleic acid molecules encoding an HA binding peptide may be incorporated in a known manner into an appropriate expression vector which ensures expression of the protein. Possible expression vectors include but are not limited to cosmids, plasmids, or modified viruses (e.g. replication defective retroviruses, adenoviruses and adeno-associated viruses). The vector should be compatible with the host cell used.

Illustrative methods of preparing HA binding peptides have been described. For example, PCT published patent application no. WO 93/21312 describes short peptides of nine or ten amino acid residues which mimic the HA binding motif of RHAMM. These RHAMM peptides possess the ability to bind HA and share a common generic peptide sequence. PCT published patent application no. WO 97/24111 also describes HA-binding peptides consisting of dextrorotatory, D-amino acids and their ability to bind naturally occurring hyaluronic acid in the body which prevents hyaluronic acid from stimulating its receptors. Correspondingly, through the inhibition of hyaluronic acid receptor activation, said D-forms of HA-binding peptides were speculated to be useful when combined with a second medicine or therapeutic agent such as a surfactant for the treatment of herpes infection, an anti-microbial agent for the treatment of mononucleosis, dimethyl sulphoxide for AIDS therapy, insulin for the treatment of diabetes and a calcium channel blocker for the treatment of hypertension.

An agent that binds an HA receptor may be any type of molecule, naturally-occurring or synthetic, that is capable of binding an HA receptor and interfering with binding of HA to its HA receptors. For example, an agent that binds an HA receptor may be a peptide, small chemical, or polynucleotide that mimics an HA oligomer. Various examples of a peptide that binds an HA receptors are provided in Ziebell et al., Chemistry & Biology 8 (2001) 1081-1094 and in Ziebell and Prestwich, Journal of Computer-Aided Molecular Design 18: 597-614, 2004, both of which are incorporated herein by reference. An agent that binds an HA receptor may be used in the treatment or prophylaxis of cancer, for example by interfering with binding of an HA receptor with HA fragment sizes that may be associated with promotion of migration, growth or survival of cancerous cells.

Dosage ranges for the administration of HA as well as agents that bind HA or HA receptors are well known to the skilled person. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications.

The dose, schedule of doses and route of administration may be varied, whether oral, nasal, vaginal, rectal, extraocular, intramuscular, intracutaneous, subcutaneous, or intravenous, and the like.

HA as well as agents that bind HA or HA receptors can be used therapeutically in combination with a pharmaceutically acceptable carrier. Pharmaceutical carriers are known to those skilled in the art. These most typically would be standard carriers for administration of compositions to humans, including solutions such as sterile water, saline, and buffered solutions at physiological pH.

Pharmaceutically acceptable carriers include liquid carriers, solid carriers, or both. Liquid carriers include, but are not limited to, water, saline, physiologically acceptable buffers, aqueous suspensions. oil emulsions, water in oil emulsions, water-in-oil-in-water emulsions, site-specific emulsions, long-residence emulsions, sticky-emulsions, microemulsions and nanoemulsions. Examples of aqueous carriers include water, saline and physiologically acceptable buffers. Examples of non-aqueous carriers include a mineral oil or a neutral oil including, but not limited to, a diglyceride, a triglyceride, a phospholipid, a lipid, an oil and mixtures thereof. Solid carriers are biological carriers, chemical carriers, or both and include, for example, particles, microparticles, nanoparticles, microspheres, nanospheres, minipumps, bacterial cell wall extracts, and biodegradable or non-biodegradable natural or synthetic polymers that allow for sustained release of the composition

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

A composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The compositions will be administered according to standard procedures used by those skilled in the art.

Compositions comprising HA of defined sizes may be used for various therapeutic and diagnostic purposes. Without wishing to be limited by theory, HA fragments accumulate during wound repair and tumour progression and modify cell behaviour by binding to HA receptors such as CD44 and Rhamm. HA fragments of a surprisingly narrow size range (10-50-mer) stimulate cell migration. For example, 30-mer HA fragments (˜6 kDa) stimulate migration of dermal fibroblasts. Slightly larger 47-mer HA fragments (˜9 kDa) stimulate migration of prostate cancer cells, while larger fragments stimulate anchorage independent growth of cancer cells. Therefore, HA fragments of defined size or compounds (peptides, small chemicals, polynucleotides) mimicking these HA fragments or their receptors, may be used to modify cell behaviour during wound repair and/or tumor progression.

Illustrative examples of use of HA, HA binding peptides or mimicking compounds thereof are now described.

Wound Repair

Purified HA of defined sizes, peptides or other chemical compounds that either mimic HA or their binding peptides could be applied topically to skin wounds. For example, HA, its binding peptides or chemical compounds that mimic HA or its binding peptides may be mixed with a gel or cream containing other active components such as collagen.

HA having a size ranging from 10 to 40, or from 10 to about 30 saccharides will be useful for wound management. Peptides that bind HA fragments that promote migration and/or growth of cancerous may also be included in wound management methods.

Any conventional method of wound management, for example topical administration or immobilization on a medical implant or diffusion from a medical implant, may be adapted to use HA, its binding peptides or mimicking compounds thereof, that are described herein. For example, U.S. Pat. No. 4,141,973, which issued on Feb. 27, 1979, discloses an ultra-pure HA fraction that has, among its many uses, the prevention of scar tissue formation and adhesion following surgery by introducing an HA solution into the surgical site, either during surgery or postoperatively. U.S. Pat. No. 5,234,914, which issued on Aug. 10, 1993, teaches the use of an HA solution for the treatment of hemorrhoids and anorectal diseases which are accompanied by traumatized tissue. The HA solution may contain between 0.01-25.0% HA, which is applied topically to the effected tissues. By adhering to the anorectal epithelium and rectal mucosa, the HA solution provides a reduction in the pain, burning, inflammation, itching, and swelling associated with the above causes. U.S. Pat. No. 5,190,759, which issued on Mar. 2, 1993, teaches the use of an HA solution, alone or in combination with dextran, for preventing adhesions between body tissues following surgical procedures. The HA solutions used therein contained between 0.5-6% HA. U.S. Pat. No. 5,409,904, which issued on Apr. 25, 1995, teaches the use of an HA solution, either alone or in combination with other viscoelastic substances, for preventing post-operative adhesions between healing tissues by introducing the solution into a surgical site during surgery or postoperatively. U.S. Pat. No. 5,681,825, which issued on Oct. 28, 1997 teaches the use of an HA solution into the site of the surgical operation, for facilitating surgical operations that involve the eye or eye area. U.S. Pat. No. 5,140,016, issued on Aug. 18, 1992 teaches the use of HA based compositions and improved methods for preventing adhesions during surgery. U.S. Pat. No. 6,136,341, issued Oct. 24, 2000, describes a tissue adhesive compound containing hydrolyzed Type I collagen having a molecular weight between 1,000 and 10,000, with the tissue adhesive compound used in combination with hyaluronic acid and glycosaminoglycans to speed the healing process further.

HA binding peptides can be useful for prevention of fibrosis of adult human tissues thereby eliminating clinical pathologies resulting from the malfunction of tissues due to keloids, hypertrophic scars, anatomonic strictures, intra-abdominal adhesions, cirrhosis of the liver, neurological deficits following spinal cord injury, valvular heart diseases, burn-injured joints as well as failure of anastomosis and adhesions following surgery. Accordingly, a method of wound management comprising administering an effective amount of a HA binding peptide of the invention to an animal in need thereof is provided. Pretreatment of skin wounds with HA binding peptides may be used to reduce fibroblast activity and deposition of collagen at the wound site thereby preventing wound contraction and tissue fibrosis. As mentioned above, fibrosis of adult human tissues is a serious clinical pathology which can result in malfunction of tissues due to keloids, hypertrophic scars, anatomonic strictures, intra-abdominal adhesions, cirrhosis of the liver, neurological deficits following spinal cord injury, valvular heart diseases, burn-injured joints as well as failure of anastomosis and adhesions following surgery. The application of HA binding peptides therapeutically to skin injuries can reduce or eliminate the adversities associated with tissue fibrosis during wound healing and these peptides possess important clinical utilities, both for therapeutic and for aesthetic purposes. Notable examples of surgical procedures which may benefit from the treatment with HA binding peptides include coronary balloon angioplasty (prevention of restenosis), small intestinal resections (e.g. in Crohn's Disease), surgery of the renal system (e.g. ureteral connection in renal transplants), and vascular surgery. Similarly, the application of HA binding peptides in plastic and cosmetic surgery can minimize the aesthetic consequences of hypertrophic scars and skin disfiguration. Illustrative methods of administering HA binding peptides have been described. For example, PCT published patent application no. WO 93/21312 describes short peptides of nine or ten amino acid residues which mimic the HA binding motif of RHAMM. These RHAMM peptides possess the ability to bind HA and share a common generic peptide sequence. PCT published patent application no. WO 97/24111 also describes HA-binding peptides consisting of dextrorotatory, D-amino acids and their ability to bind naturally occurring hyaluronic acid in the body which prevents hyaluronic acid from stimulating its receptors. US Patent Publication No. 2004/0034201 provides a further example of methods for administering HA binding peptides.

The methods of wound management taught in these U.S. patents may be adapted to use HA or HA binding compounds described herein.

Tissue Engineering

HA of defined sizes, peptides or mimicking compounds thereof could allow temporal and spatial control of cell type specific cell growth and/or migration during tissue engineering applications. HA fragments or other compounds could be integrated into matrices or scaffolds, which are commonly used in tissue engineering or applied to the culture medium. For example, HA having a size ranging from 10 to 40, or from 10 to about 30 saccharides will be useful for tissue engineering applications.

Maintenance of cells ex vivo is dependent on the use of nutrients, substrata of specific extracellular matrix components, and mixtures of soluble signals that include hormones and growth factors. Distinct defined mixtures of nutrients, matrix components and soluble signals elicit survival, expansion and differentiation of cells. Moreover, the composition of the defined mixtures is lineage dependent with specific compositions required for stem cells versus intermediates in the lineage versus mature cells. The mixtures complexed with HA offer a native, 3-dimensional (3-D) signaling scaffold, with an extent of solidity regulated by forms of cross-linking in addition to base matrix molecules, and all offer considerable advantages for tissue engineering ex vivo and for forms of grafts for cells to be reintroduced to animals (or people) in vivo. Such complexes are useful also for stem cells, for example, hepatic stem cells and their progeny (e.g., hepatoblasts and committed progenitors), that can be established in a complex comprised of a defined mixture of components to elicit dramatic 3-D expansion or can be seeded into ones that will drive 3-D differentiation. HA, and its binding peptides can benefit maintenance, migration, and/or growth of desirable candidates for cell-based therapies, such as stem cells or induced pluripotent cells.

HA, its binding peptides or mimicking compounds thereof may be used in accordance with conventional tissue engineering techniques. In many instances of tissue engineering a matrix may advantageously be composed of biopolymers, including polypeptides or proteins, as well as various polysaccharides, including proteoglycans and the like. In addition, these biopolymers may be either selected or manipulated in ways that affect their physico-chemical properties. For example biopolymers may be cross-linked either enzymatically, chemically or by other means, thereby providing greater or lesser degrees of rigidity or susceptibility to degradation.

Various constituents of the extracellular matrix are among the many natural polymers which have been disclosed to be useful for tissue engineering or culture, including fibronectin, various types of collagen, laminin, keratin, fibrin and fibrinogen, HA, heparin sulfate, chondroitin sulfate and others.

Many tissue engineering or cell culture techniques are available that could benefit from appropriate incorporation of HA, its binding peptides or mimicking compounds thereof described herein. For example, U.S. Pat. Nos. 5,955,438 and 4,971,954 disclose collagen-based matrices cross-linked by sugars, useful for tissue regeneration. U.S. Pat. No. 5,948,429 discloses methods of making and using biopolymer foams comprising extracellular matrix particulates. U.S. Pat. Nos. 6,083,383 and 5,411,885 disclose fibrin or fibrinogen glue and methods for using same. U.S. Pat. Nos. 5,279,825 and 5,173,295 disclose a method of enhancing the regeneration of injured nerves and adhesive pharmaceutical formulations comprising fibrin. U.S. Pat. No. 4,642,120 discloses the use of fibrin or fibrinogen glue in promoting repair of defects of cartilage and bone. U.S. Pat. Nos. 6,124,265 and 6,110,487 disclose methods of making and cross-linking keratin-based films and sheets and of making porous keratin scaffolds and products of same. The utility of HA as a beneficial component for supporting tissue growth is also well established in the art, as exemplified in U.S. Pat. No. 5,942,499, which discloses methods of promoting bone growth with HA and growth factors. U.S. Pat. Nos. 5,128,326 and 5,783,691 disclose methods of producing and using cross-linked HA in promoting tissue repair and as reservoirs for bioactive agents including drugs or growth factors. International Patent Application PCT/IL99/00257 (published as WO 99/58042) discloses methods of ameliorating impairments of the central nervous system by culturing neural tissue on a matrix gel composed of HA and laminin. It was previously reported that the combination of HA and laminin provides both a flexible elastic bonding and tight rigid bonding cell matrix. Goldman et al. (Ann. N.Y. Acad. Sci. 835, 30-55, 1997) disclosed certain preliminary results using this technique.

Cancer Treatment

Peptides or other compounds that either mimic HA fragments or their receptors could be dissolved in PBS or physiologic saline solution and given to patients by i.v. injection, i.m. injection or orally. Following surgical removal of the tumor, HA fragments, peptides or other compounds could be applied directly to the surgical wound bed to stimulate wound repair and/or prevent proliferation and invasion of remaining tumor cells.

Locomotion and motility of tumour cells are fundamental to their ability to invade tissues and/or metastasize. The ability of HA binding peptides to inhibit the migration and/or growth of cancerous cells implicates their effectiveness in preventing tumour metastasis and their utility as cancer chemotherapeutic agents. Accordingly, a method of preventing or reducing tumour metastasis comprising administering an effective amount of a HA binding peptide or a nucleic acid molecule encoding a HA binding peptide of the invention to an animal in need thereof is provided herein.

Diagnostics

Imaging agents may be produced using HA or HA binding peptides, or chemical compounds that mimic HA or HA binding peptides.

HA fragments accumulate during malignancies such as breast and prostate cancer in the peritumoural stroma and inside tumor cells and during wound repair in the reactive stroma/granulation tissue.

Labelled peptides or other chemical compounds that mimic HA receptors or HA binding peptides could be used to image these tissue areas of increased HA fragment accumulation. Peptides could be modified to create an imaging probe by incorporating fluorophores such as FITC/TRITC, near-infrared optical dyes such as Cy5.5, gamma emitting radioisotopes such as technetium-99m for SPECT (single photon emission computed tomography), positron emitting radioisotopes such as fluorine-18 for PET (positron emission tomography), or contrast agents such as chelated gadolinium.

HA or chemical compounds that mimic HA could be used to image HA receptor expression or HA binding activity within biological samples. HA or chemical compounds that mimic HA could be modified to create an imaging probe by incorporating fluorophores such as FITC/TRITC, near-infrared optical dyes such as Cy5.5, gamma emitting radioisotopes such as technetium-99m for SPECT (single photon emission computed tomography), positron emitting radioisotopes such as fluorine-18 for PET (positron emission tomography), or contrast agents such as chelated gadolinium.

These imaging agents may be administered by i.v. injection. Expression of HA receptors such as CD44 and Rhamm/HMMR is upregulated during tumor progression and wound repair. Labelled HA fragments of defined size, peptide or other chemical compounds that mimic these fragments could be used to image tumors and wound repair. HA fragments, peptides or other compounds could be labelled and administered as described above for in vivo imaging techniques such as magnetic resonance imaging or PET. Imaging of biological samples in vitro is also contemplated.

Techniques of imaging other than directly labeling HA or HA binding peptides, or chemical compounds that mimic HA or HA binding peptides are available to the skilled person. For example, compounds that recognize and bind HA and/or HA binding peptide. In certain examples, any type of antibody may be used. A polyclonal or a monoclonal antibody purified by affinity chromatography with single epitope, or a monoclonal antibody capable of binding efficiently with HA binding protein are examples of useful antibodies. When a polyclonal antibody is used to determine HA/protein interactions, the antibody can be prepared by a conventional method of immunization of an animal such as horse, cow, sheep, rabbit, goat, rat or mouse with an HA binding protein. A monoclonal antibody can also be prepared according to a conventional method, for example, using a hybridoma cell obtained by fusing a cell line derived from mouse myeloma with cells from the mouse spleen which is preliminarily immunized with an HA binding protein.

Antibodies against HA itself are also available. For example, GenWay (San Diego, USA) sells a sheep polyclonal antibody raised against HA.

Antibodies may be supported on any one of carriers usually used in the immunological measurement, for example, carriers prepared from natural organic polymer substances such as red blood cell, bacteria, cell fragment and the like, assembly of molecule such as liposome, polymeric micelle and the like, synthetic polymer compounds such as polystyrene, polyacrylic acid, polymethacrylic acid, polyacrylamide, polyglycidylmethacrylate, polypropylene, polyvinylchloride, polyethylene, polychlorocarbonate, silicone resin, silicone rubber and the like, inorganic substances such as porous glass, ground glass, alumina, silica gel, activated carbon and metal oxide are included. In addition, these carriers can be used in various forms such as tube, bead, disc type chip, micro particle or latex particle.

The method of supporting an antibody involved in determining HA/protein interactions on a carrier may be performed without specific limitation. All supporting methods well known per se usually used in this field can be included, for example, supporting an antibody on the carrier by physical adsorption or chemical linkage.

Methods for quantifying antigen/antibody interactions are well known, and any such conventional method may be used.

A diagnostic kit for determining HA interactions with HA binding peptides or receptors can comprise one or more of imaging agents derived from HA, an HA binding peptide, or a compound that mimics HA or HA binding peptides, or an antibody that recognizes HA and/or an HA binding peptide.

Components of the kit may be dissolved in an appropriate buffer solution. As the buffering agents used for this purpose, any kind of buffering agent usually used in the immunological measurement, for example, Tris buffering agent, phosphate buffering agent, veronal buffering agent, boric acid buffering agent and Good buffering agent can be adopted, and the concentration of such buffering agent is usually 5 to 300 mM, preferably 10 to 150 mM, and the pH is usually 5 to 10, preferably 6 to 8, and the concentration and pH are each selected appropriately from above described corresponding range. Components of the kit may be prepared with any other substance for enhancing the shelf life of the kit, for example, a stabilizing agent such as a sugar, a protein and a surface activating agent, a salt such as NaCl or an preservative substance and the like may be added as desired.

The diagnostic kits may be used in accordance with conventional diagnostic methods and will typically involve imaging of a live subject, for example using labeled HA or labeled HA binding peptides administered to the subject, or may involve a biological sample obtained from a subject, for example, a body tissue, such as breast or prostate biopsies, or any body tissue suspected of containing a tumorous growth, or a body fluid such as blood, plasma, serum, synovial fluid, pleural fluid, lymph fluid, spinal fluid and urine.

Screening Methods

HA, HA binding peptides, or mimicking compounds thereof may be used in screening methods. For example, a method of identifying a cancer cell that is susceptible to treatment using an HA depletion technique, for example using an HA binding peptide, comprises culturing a cancer cell in the presence of an HA and determining whether migration and/or proliferation of the cancer cell increases in the presence of the HA. The cancer cell may be from a biological sample, such as a patient biopsy, or may be an established cell line, such as commercially available cell lines or those maintained by American Type Culture Collection (ATCC) or other biological deposit authority. The HA used in the screening of cancer cells will typically be 40 mer to 100 mer or of the other sizes described herein as promoting cancerous migration or growth.

When introducing elements disclosed herein, the articles “a”, “an”, “the”, and “said” are intended to mean that there are one or more of the elements unless the context dictates otherwise. For example, the term “a compound” and “at least one compound” may include a plurality of compounds, including mixtures thereof. The terms “comprising”, “having”, “including” are intended to be open-ended and mean that there may be additional elements other than the listed elements. The phrase “consisting essentially of” is intended to be limiting to specified elements and those further elements that do not materially affect the basic and novel characteristic of the combination of specified elements. For example, a composition defined using the phrase “consisting essentially of” encompasses any known pharmaceutically acceptable additive, excipient, diluent, carrier, and the like.

The above detailed description is solely for purposes of illustration and is not intended to limit the scope of the claims. A more complete understanding can be obtained by reference to the following specific Examples. The Examples are also described solely for purposes of illustration and are not intended to limit the scope of the claims. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Peptide Structures that Selectively Bind HA

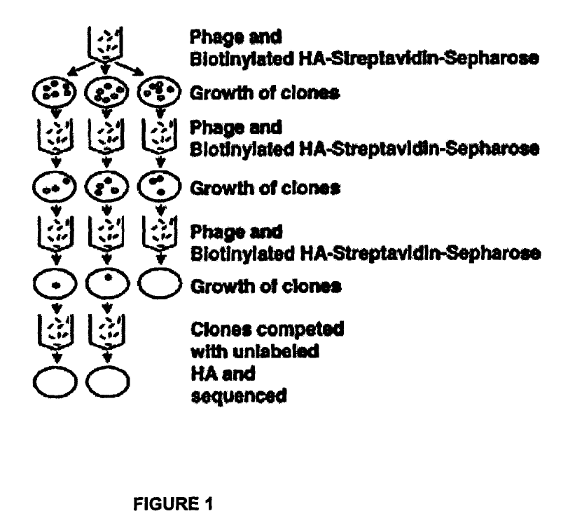

Interfering with HA/tumor cell interactions may be a useful approach for developing new therapies in prostate and breast cancer. Synthetic peptide structures that selectively bind HA, can be used for interfering with HA-mediated growth, survival or invasion. A phage display approach may be used for identifying synthetic peptides, as outlined in FIG. 1.

Briefly, the phage display approach utilized a 15mer phage library (Chiron). Biotinylated HA (heterogeneous molecular weight) was coupled to streptavidin sepharose beads, and the beads were then mixed with phage. After washing, bound phage was eluted with 100 microgram/ml of HA and the recovered phage plated onto a bacterial lawn. This selection was repeated 3 times. Negative controls included testing the isolated phage for failure to bind either streptavidin or biotin-sepharose.

Using this phage display approach, 65 phage clones were isolated, 47 of which contained the primary sequence (STMMSRSHKTRSHHV; peptide 15-1; the BX7B motif is underlined). This sequence contains a BX7B motif similar to other HA binding proteins such as HABP-1, HABP-4 and CDC-37 [26-28] and it is similar in structure to another HA binding peptide used to inhibit HA function during lung repair [29]. Eighteen copies of a second clone (TMTRPHFHKRQLVS; peptide 15-2; the BX7B-like motif is underlined), which contained part of this sequence, were also isolated using the phage display procedure. Additional phage libraries (7-mer and 12-mer) were also used, resulting in the identification of additional HA-binding peptides with a structural motif (F/WHKP/A; i.e., F or W followed by HK followed by P or A) that represents a minimal portion of the BX7B HA binding motif (not shown). Peptide structures isolated from these additional libraries tended to have a higher representation of hydrophobic amino acids than the 15-mer.

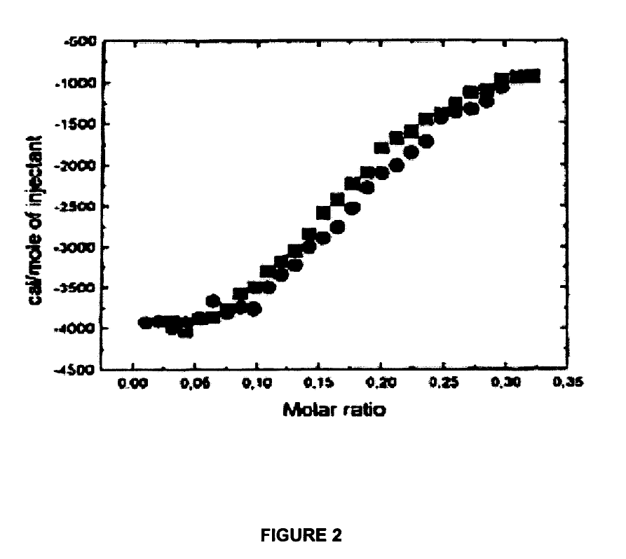

The binding of soluble peptide 15-1 to HA was next evaluated by isothermal calorimetry (ITC), to estimate a binding constant for the peptide (FIG. 2, the two lines represent two independent determinations in the figure). A buffered solution of 0.9 mM low molecular weight HA (10 kD average molecular weight) was injected into to a 0.6 mM peptide 15-1 solution. The binding curve is sigmoidal and saturable, with an estimated moderate binding constant (Kd) of 10⁻⁷ M. A scrambled peptide (HKSVSRHTSMRHSTM) used as a control did not interact with HA under these conditions. Furthermore, when high molecular weight HA was used instead of fragmented HA, the interaction of the 15-1 peptide was greatly reduced (not shown). The results suggest that the primary sequence of peptide 15-1 contains a structural motif that binds low molecular weight HA with high affinity, but its affinity to high molecular weight HA being comparatively lower. This peptide (and the scrambled control) have been further characterized in biological systems.

### Example 2

### Peptide 15-1 Inhibition of HA Stimulated Motility

Scratch wound assays with Rhamm null and wildtype fibroblast cells were used to evaluate the biological properties of peptide 15-1.

Scratch wound assays are commonly used to assess the effects of drugs and drug candidates on cellular migration associated with wound closing. In a typical scratch wound assay, cells are plated to confluence in a multiwell plate. A single scratch wound is created in each well. The plate is then imaged at fixed time intervals. Cell number in the area of the scratch wound can be quantified to evaluate the characteristics of wound closing in the presence of pharmacological agents.

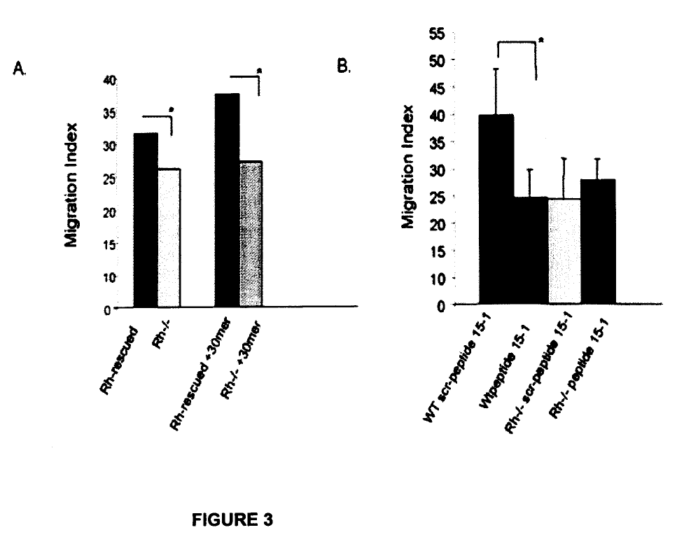

Using the scratch wound assay, Rhamm null cells exhibited less migration in response to HA compared to wild type cells, and the migration of Rhamm null cells was resistant to the inhibitory effects of the peptide 15-1 (FIG. 3). The lack of HA induced migration was reversed by re-expression of Rhamm (FIG. 3a). HA stimulated migration of wildtype cells, but not Rh−/−, cells is sensitive to the inhibitory effects of the synthetic HA binding peptide 15-1 (FIG. 3b).

### Example 3

### Effect of HA Binding Peptide 15-1 on Tumor Growth

A methylcellulose assay was adapted to evaluate anchorage-independent growth of prostate tumor cells in vitro. This assay offers advantages over agarose since the gels can be easily solubilized, allowing for recovery and quantification/biochemical characterization of cells at the end of the experiment. Prostate tumor PC3M-LN4 cells which synthesize large amounts of HA grow in this assay to form large multicellular colonies. Cells were recovered and counted after 7 days of incubation.

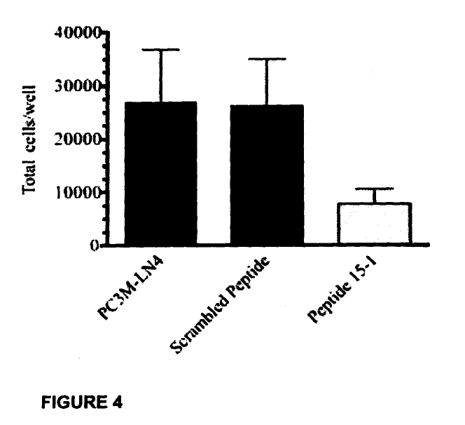

Fifteen thousand PC3M-LN4 cells were seeded into methylcellulose in the presence of 50 micromolar of the HA binding peptide 15-1 or scrambled peptide. Each peptide was mixed with PC3M-LN4 cells prior to culturing these cells in methylcellulose (FIG. 4). Culturing these cells in the presence of 50 micromolar peptide 15-1 inhibits anchorage-independent growth by 80%, which is highly statistically significant (FIG. 4).

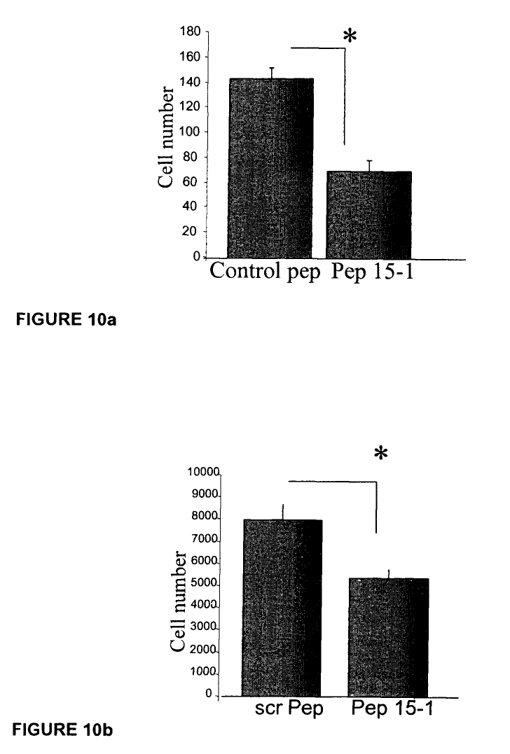

The efficacy of peptide 15-1 in reducing migration of breast cancer cells has also been demonstrated. FIG. 10a shows that peptide 15-1 reduces scratch wound induced migration of human breast cancer cells. A confluent monolayer of MDA-MB-231 cells were scratched using a pipette tip. Cells were incubated in medium containing either peptide 15-1 (STMMSRSHKTRSHHV) or scrambled control peptide (HKSVSRHTSMRHSTM) (30 ug/ml). After 24 hrs, cells that migrated into the scratch were counted.

Peptide 15-1 has also been shown to reduce anchorage independent growth of human breast cancer cells. MDA-MB 231 cells were grown in Methylcellulose containing either peptide 15-1 (STMMSRSHKTRSHHV) or scrambled control peptide (HKSVSRHTSMRHSTM) (30 ug/ml). One week later, cells were counted. Results are shown in FIG. 10b,

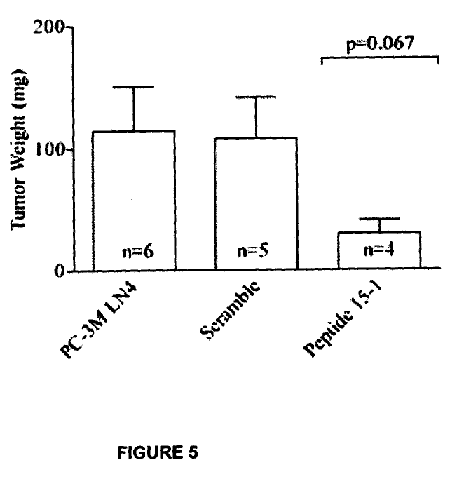

Studies have also been performed to determine if peptide 15-1 can inhibit tumor growth in a more complex microenvironment (FIG. 5). One hundred thousand tumor cells pretreated with 50 micromolar peptide 15-1 were injected subcutaneously into NOD.CB17 SCID mice. Twenty eight days later the mice were euthanized and the tumors harvested and analyzed. Peptide pretreatment results in a decrease in tumor growth.

### Example 4

### Effects of HA Fragment Size Variance

Experiments were performed to assess whether or not different sized HA fragments exert different effects on cell behavior. Highly purified, sized HA fragments from Seikagku (4-mer, 6-mer, 8-mer, 10-mer 12-mer and 30-mer) and LifeCore (4167-mer, 1145-mer, 260-mer, 192-mer, 114-mer and 47-mer) and quantified the consequences of these sized fragments on anchorage independent growth in 3 dimensional culture and cell migration in scratch wound assays in culture were quantified.

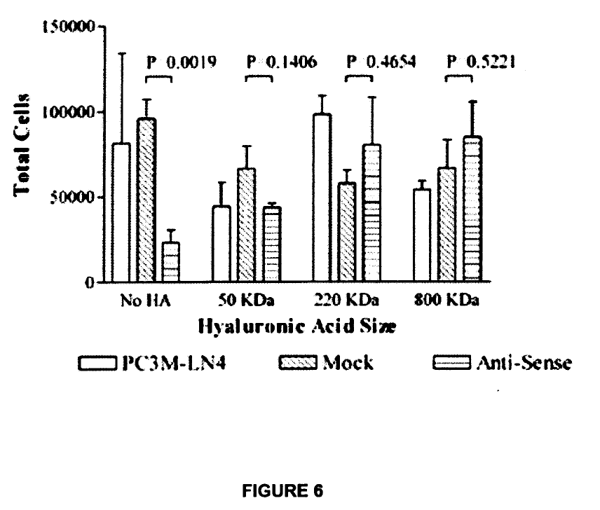

A methylcellulose assay was adapted to evaluate anchorage-independent growth of prostate tumor cells in vitro. This assay offers advantages over agarose since the gels can be easily solubilized, allowing for recovery and quantification/biochemical characterization of cells at the end of the experiment. Prostate tumor PC3M-LN4 cells which synthesize large amounts of HA grow in this assay to form large multicellular colonies. After 7 days of incubation, the cells were recovered and counted (FIG. 6). PC3M-LN4 cells and mock-transfectants plated at low density (3×104/culture) within these matrices exhibit anchorage-independent growth over the 7 days of the assay (FIG. 6). Inhibiting HA synthesis with anti-sense molecules targeting HA synthases (HAS2 and HAS3) causes a significant inhibition of growth that can be reversed by the addition of highly purified HA (LifeCore, Chaska, Minn.). Reversal of growth inhibition is most notable in the presence of higher molecular weight HA (220 and 800 kD), and to a lesser extent 50 kD HA. 0.66 mg/mL of the various HA sizes were used in this assay.

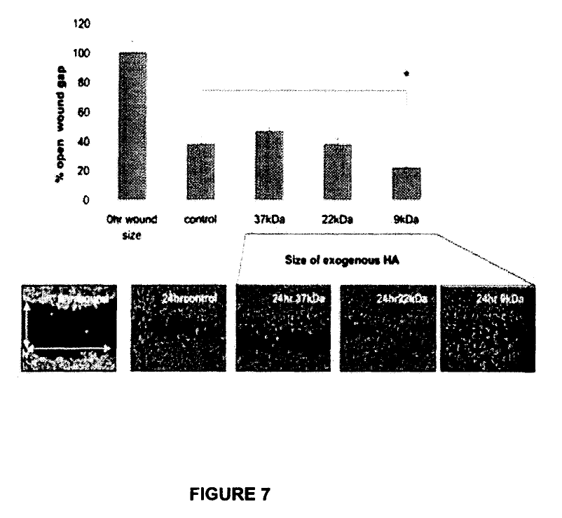

The ability of various sizes of HA oligomers to promote migration of prostate tumor PC3M-LN4 cells was tested. For these assays, the scratch wound assay supplemented (or not) with various sizes of HA oligomers was used. As can be seen in FIG. 7, the rate of wound closure/motility (expressed as the size of each wound remaining to be closed) is enhanced in the presence of purified 9 kD (47-mer) oligomeric HA, whereas larger 22 and 37 kD (114 and 192-mer, respectively) fragments are either ineffective or inhibitory for promoting wound closure. Several smaller and larger oligomers (2.5 kD to 6 kD, 220 and 880 kD) may also be tested for comparison.

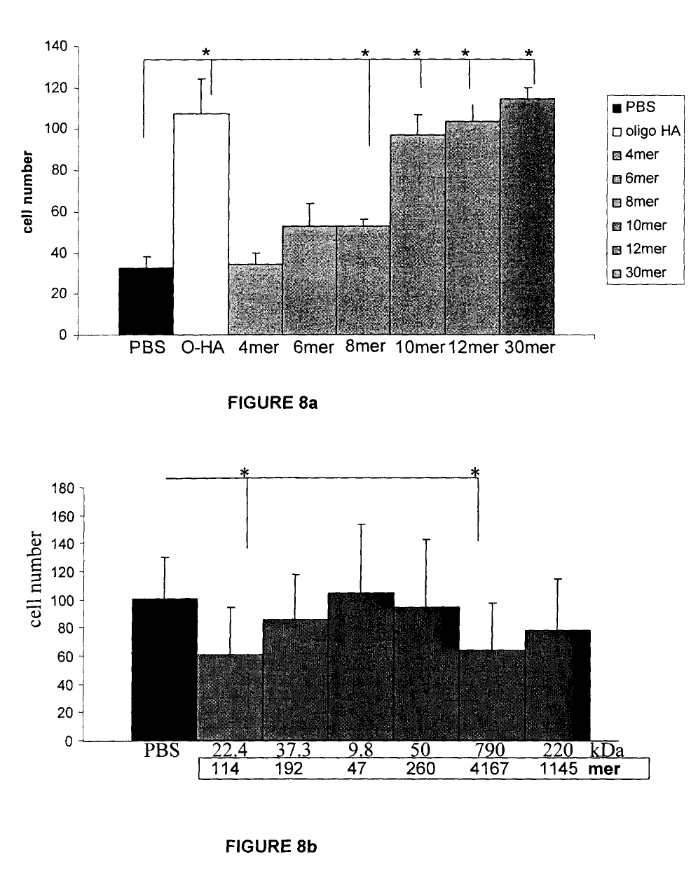

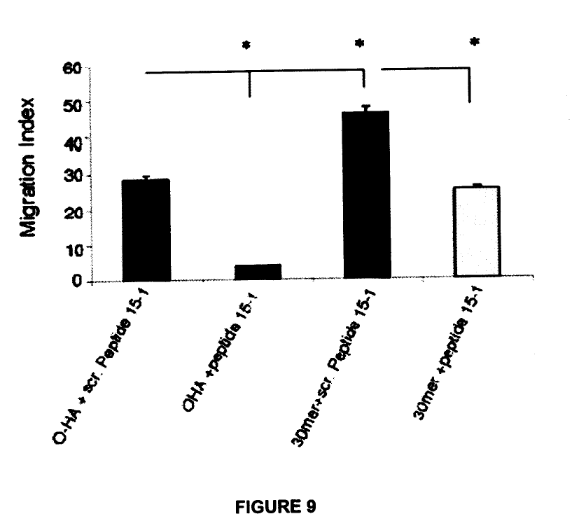

The scratch wound assay was also used to determine the ability of various sizes of HA oligomers to promote migration of wild-type dermal fibroblast cells (FIGS. 8a and 8b). As seen in FIG. 8a, only the 10-mer, 12-mer and 30-mer HA fragments stimulated dermal fibroblast migration, with the 30-mer (5.7 kDa) HA fragment consistently and most strongly stimulating migration. Scratch wound assays were also used to show that the stimulatory effect of the 30-mer HA fragment was inhibited by a peptide (peptide 15-1) that binds to HA fragments but was not inhibited by a scrambled counterpart (scr. Peptide 15-1) of this peptide which is unable to specifically bind HA (FIG. 9).

These results indicate that different sizes of HA fragments exert different biological effects for wild-type versus transformed/cancerous cell types.

### REFERENCES

The disclosures of all references and documents cited throughout the specification are incorporated by reference herein.
1. Hascall, V. C., Hyaluronan, a common thread. Glycoconj J, 2000. 17(7-9): p. 607-16.
2. Salustri, A., et al., Hyaluronan and proteoglycans in ovarian follicles. Hum Reprod Update, 1999. 5(4): p. 293-301.
3. Tammi, M. I., A. J. Day, and E. A. Turley, Hyaluronan and homeostasis: a balancing act. J Biol Chem, 2002. 277(7): p. 4581-4.
4. Toole, B. P., Hyaluronan: from extracellular glue to pericellular cue. Nat Rev Cancer, 2004. 4(7): p. 528-39.
5. Itano, N. and K. Kimata, Mammalian hyaluronan synthases. IUBMB Life, 2002. 54(4): p. 195-9.
6. Spicer, A. P. and J. A. McDonald, Characterization and molecular evolution of a vertebrate hyaluronan synthase gene family. J Biol Chem, 1998. 273(4): p. 1923-32.
7. Spicer, A. P., et al., Chromosomal localization of the human and mouse hyaluronan synthase genes. Genomics, 1997. 41(3): p. 493-7.
8. Day, A. J. and G. D. Prestwich, Hyaluronan-binding proteins: tying up the giant. J Biol Chem, 2002. 277(7): p. 4585-8.
9. Lee, J. Y. and A. P. Spicer, Hyaluronan: a multifunctional, megaDalton, stealth molecule. Cum Opin Cell Biol, 2000. 12(5): p. 581-6.
10. Toole, B. P., T. N. Wight, and M. I. Tammi, Hyaluronan-cell interactions in cancer and vascular disease. J Biol Chem, 2002. 277(7): p. 4593-6.
11. Cho, R. J., et al., Transcriptional regulation and function during the human cell cycle. Nat Genet, 2001. 27(1): p. 48-54.
12. Brecht, M., et al., Increased hyaluronate synthesis is required for fibroblast detachment and mitosis. Biochem J, 1986. 239(2): p. 445-50.
13. Evanko, S. P. and T. N. Wight, Intracellular localization of hyaluronan in proliferating cells. J Histochem Cytochem, 1999. 47(10): p. 1331-42.
14. Mohapatra, S., et al., Soluble hyaluronan receptor RHAMM induces mitotic arrest by suppressing Cdc2 and cyclin B1 expression. J Exp Med, 1996. 183(4): p. 1663-8.
15. Ghatak, S., S. Misra, and B. P. Toole, Hyaluronan oligosaccharides inhibit anchorage-independent growth of tumor cells by suppressing the phosphoinositide 3-kinase/Akt cell survival pathway. J Biol Chem, 2002. 277(41): p. 38013-20.
16. Yu, Q., B. P. Toole, and I. Stamenkovic, Induction of apoptosis of metastatic mammary carcinoma cells in vivo by disruption of tumor cell surface CD44 function. J Exp Med, 1997. 186(12): p. 1985-96.
17. Lipponen, P., et al., High stromal hyaluronan level is associated with poor differentiation and metastasis in prostate cancer. Eur J Cancer, 2001. 37(7): p. 849-56.
18. Lokeshwar, V. B., et al., Association of elevated levels of hyaluronidase, a matrix-degrading enzyme, with prostate cancer progression. Cancer Res, 1996. 56(3): p. 651-7.
19. Lokeshwar, V. B., et al., Stromal and epithelial expression of tumor markers hyaluronic acid and HYAL1 hyaluronidase in prostate cancer. J Biol Chem, 2001.276(15): p. 11922-32.
20. Aaltomaa, S., et al., Strong Stromal Hyaluronan Expression Is Associated with PSA Recurrence in Local Prostate Cancer. Urol Int, 2002. 69(4): p. 266-72.
21. Kovar, J. L., et al., Hyaluronidase expression induces prostate tumor metastasis in an orthotopic mouse model. Am J Pathol, 2006. 169(4): p. 1415-26.
22. Simpson, M. A., Concurrent expression of hyaluronan biosynthetic and processing enzymes promotes growth and vascularization of prostate tumors in mice. Am J Pathol, 2006. 169(1): p. 247-57.
23. Ekici, S., et al., Comparison of the prognostic potential of hyaluronic acid, hyaluronidase (HYAL-1), CD44v6 and microvessel density for prostate cancer. Int J Cancer, 2004. 112(1): p. 121-9.
24. Posey, J. T., et al., Evaluation of the prognostic potential of hyaluronic acid and hyaluronidase (HYAL1) for prostate cancer. Cancer Res, 2003. 63(10): p. 2638-44.
25. McCarthy, J. B. and M. A. Simpson, Hyaluronan in Prostate Cancer Progression. Hyaluronan Today (electronic publication http://www.glycoforum.gr.jp/), 2003.
26. Chen, Q., et al., Spacrcan binding to hyaluronan and other glycosaminoglycans. Molecular and biochemical studies. J Biol Chem, 2004. 279(22): p. 23142-50.
27. Deb, T. B. and K. Datta, Molecular cloning of human fibroblast hyaluronic acid-binding protein confirms its identity with P-32, a protein co-purified with splicing factor SF2. Hyaluronic acid-binding protein as P32 protein, co-purified with splicing factor SF2. J Biol Chem, 1996. 271(4): p. 2206-12.
28. Huang, L., et al., Molecular characterization of a novel intracellular hyaluronan-binding protein. J Biol Chem, 2000. 275(38): p. 29829-39.
29. Savani, R. C., et al., A role for hyaluronan in macrophage accumulation and collagen deposition after bleomycin-induced lung injury. Am J Respir Cell Mol Biol, 2000. 23(4): p. 475-84.

1-93. (canceled)

94. A composition comprising a first HA oligomer of a size of 10-mer to about 40-mer in higher concentration than any other size of HA oligomer.

95. The composition of claim 94, wherein the first HA oligomer is selected from a 10-mer to about 35-mer or a 10-mer to about 30-mer.

96. The composition of claim 95, wherein the first HA oligomer preferentially promotes migration of a wild-type cell over a cancerous cell.

97. The composition of claim 96, wherein the first HA oligomer is a mixture of a plurality of sizes, or a single size.

98. The composition of claim 94, wherein a 47-mer HA oligomer or a HA oligomer of greater than 100000 dalton or a HA oligomer greater than and equal to a size of 47-mer is not present.

99. The composition of claim 94, further comprising an agent that binds to a 47-mer HA oligomer.

100. A method of promoting migration of a wild-type cell in a subject comprising administering an effective amount of a composition of claim 94 to a subject in need of such treatment.

101. The method of claim 100, wherein the first HA oligomer is selected from a 10-mer to about 35-mer or a 10-mer to about 30-mer.

102. The method of claim 100, wherein the first HA oligomer preferentially promotes migration of a wild-type cell over a cancerous cell.

103. The method of claim 100, wherein the first HA oligomer is a mixture of a plurality of sizes or is a single size.

104. The method of claim 100, wherein a 47-mer HA oligomer or a HA oligomer of greater than 100000 dalton is not present or a HA oligomer greater than and equal to a size of 47-mer is not present.

105. The composition of claim 94, incorporated into one or more of a nutritional supplement, a suture, a bandage, a transdermal patch or a capsule.

106. The composition of claim 94 provided as an injectable, topical or oral pharmaceutical composition.

107. The composition of claim 94, formulated to promote migration of fibroblasts and/or epithelial cells.

108. A composition comprising a first HA oligomer of a size of about 40-mer to about 80-mer in higher concentration than any other size of HA oligomer.

109. The composition of claim 108, wherein the first HA oligomer is selected from about 40-mer to about 60-mer or about 45-mer to about 50-mer.

110. The composition of claim 108, wherein the first HA oligomer preferentially promotes migration of a cancerous cell over a wild-type cell.

111. The composition of claim 108, wherein the first HA oligomer is a mixture of a plurality of sizes or a single size.

112. The composition of claim 108, wherein the first HA oligomer is a 47-mer HA oligomer.

113. The composition of claim 108, wherein an HA oligomer of greater than 100000 dalton is present.

114. The composition of claim 108, further comprising a 1145-mer HA oligomer and/or a 4167-mer HA oligomer.

115. A method of promoting migration of a cancerous cell in a subject comprising administering an effective amount of a composition of claim 108 to a subject in need of such treatment.

116. The method of claim 115, wherein the first HA oligomer is about 40-mer to about 60-mer or about 45-mer to about 50-mer.

117. The method of claim 115, wherein the first HA oligomer preferentially promotes migration of a cancerous cell over a wild-type cell.

118. The method of claim 115, wherein the first HA oligomer is a mixture of a plurality of sizes, or is a single size.

119. The method of claim 115, wherein the first HA oligomer is a 47-mer HA oligomer.

120. The method of claim 115, wherein an HA oligomer of greater than 100000 dalton is present.

121. The method of claim 115, further comprising an 1145-mer HA oligomer.

122. The method of claim 115, further comprising a 4167-mer HA oligomer.

123. A method of imaging a cancerous cell in a subject comprising, administering an imaging effective amount of the composition of claim 108 to a subject, wherein the first HA oligomer is labeled; and scanning a target area of the subject to determine the presence of the first labeled HA oligomer within the target area.

124. The method of claim 123, wherein the first HA oligomer is radio-labelled or fluorescent-labelled.

125. A method of diagnosing a cancer in a subject comprising, administering a diagnostic effective amount of the composition of claim 108, to a subject, wherein the first HA oligomer is labeled; and determining whether the first labeled HA oligomer binds to a cell in the subject.

126. The method of claim 125, wherein the first HA oligomer is radio-labelled or fluorescent-labelled.

127. A composition comprising a peptide that binds an HA oligomer of a size of about 10-mer to about 80-mer.

128. The composition of claim 127, wherein the peptide comprises the sequence n-terminal-STMMSRSHKTRSHHV-c-terminal.

129. A method of treating cancer in a subject comprising administering an effective amount of the composition of claim 127 to a subject in need of such treatment.

130. A method of treating cancer in a subject comprising administering an effective amount of the composition of claim 128 to a subject in need of such treatment.

131. A composition comprising a peptide that binds to an HA binding domain of an HA receptor.

132. The composition of claim 131, wherein the HA binding domain binds to an HA oligomer of a size of about 10-mer to about 80-mer.

133. A method of treating cancer in a subject comprising administering an effective amount of the composition of claim 132 to a subject in need of such treatment.

134. An HA oligomer selected from an oligomer having a size from 10 to about 40 saccharides; or a size from 10 to about 30 saccharides.

135. The HA oligomer of claim 134, wherein the HA oligomer preferentially promotes migration of a wild-type cell over a cancerous cell.

136. The HA oligomer of claim 134, wherein the HA oligomer is labelled.

137. The HA oligomer of claim 136, wherein the HA oligomer is radio-labelled or fluorescent-labelled.

138. An HA oligomer having a size selected from about 40 to about 80 saccharides from about 45 to about 50 saccharides; or a size of about 47 saccharides.

139. The HA oligomer of claim 138, wherein the HA oligomer preferentially promotes migration of a wild-type cell over a cancerous cell.

140. The HA oligomer of claim 138, wherein the HA oligomer is labelled.

141. The HA oligomer of claim 138, wherein the HA oligomer is radio-labelled or fluorescent-labelled.

142. A method of determining HA levels in a biological sample comprising contacting the biological sample with the composition of claim 127, and quantifying the peptide/HA interaction.

143. A method of determining HA levels in a biological sample comprising contacting the biological sample with the composition of claim 128, and quantifying the peptide/HA interaction.

144. A method of imaging HA in a biological sample comprising contacting the biological sample with the composition of claim 127, and imaging the peptide, wherein the peptide is labelled.

145. A method of imaging HA in a biological sample comprising contacting the biological sample with the composition of claim 128, and imaging the peptide, wherein the peptide is labelled.

146. A screening method for identifying a cancer cell that exhibits HA-dependent migration and/or growth, comprising culturing a cancer cell in the presence or absence of the HA oligomer of claim 138, and determining whether migration and/or proliferation of the cancer cell increases in the presence of the HA.

147. The screening method of claim 146, wherein the cancer cell is from a biological sample obtained from a patient.

148. The screening method of claim 146, wherein the cancer cell is an established cell line.
